(19) United States
(12) Patent Application Publication
(10) Pub. No.: US 2015/0274819 A1
(43) Pub. Date: Oct. 1, 2015

(54) STABLE AQUEOUS RECOMBINANT PROTEIN FORMULATIONS

(71) Applicants: LA JOLLA BIOLOGICS, INC., San Diego, CA (US); TANVEX BIOLOGICS, CORPORATION, New Taipei City (TW)

(72) Inventor: Shiang GWEE, Oceanside, CA (US)

(21) Appl. No.: 14/636,083

(22) Filed: Mar. 2, 2015

Related U.S. Application Data
(60) Provisional application No. 61/947,231, filed on Mar. 3, 2014.

(51) Int. Cl. C07K 16/22

(52) U.S. Cl. CPC C07K 16/22

(57) ABSTRACT

The present invention provides a sterile pharmaceutical formulation suitable for parenteral administration comprising: (a) a recombinant protein; (b) sodium acetate; (c) arginine hydrochloride; (d) a cryoprotecant; and (e) a surfactant.

### CROSS-REFERENCE TO RELATED APPLICATIONS

This application claims the benefit of U.S. Provisional Application No. 61/947,231, filed Mar. 3, 2014, which is hereby incorporated in its entirety including all tables, figures, and claims.

### FIELD OF THE INVENTION

The present invention relates to stable aqueous recombinant protein formulations suitable for parenteral administration.

### BACKGROUND OF THE INVENTION

In the past ten years, advances in biotechnology have made it possible to produce a variety of proteins for pharmaceutical applications using recombinant DNA techniques. Because proteins are larger and more complex than traditional organic and inorganic drugs (i.e. possessing multiple functional groups in addition to complex three-dimensional structures), the formulation of such proteins poses special problems. For a protein to remain biologically active, a formulation must preserve intact the conformational integrity of at least a core sequence of the protein's amino acids while at the same time protecting the protein's multiple functional groups from degradation. Degradation pathways for proteins can involve chemical instability (i.e. any process which involves modification of the protein by bond formation or cleavage resulting in a new chemical entity) or physical instability (i.e. changes in the higher order structure of the protein). Chemical instability can result from deamidation, racemization, hydrolysis, oxidation, beta elimination or disulfide exchange. Physical instability can result from denaturation, aggregation, precipitation or adsorption, for example. The three most common protein degradation pathways are protein aggregation, deamidation and oxidation. Cleland et al. Critical Reviews in Therapeutic Drug Carrier Systems 10(4): 307-377 (1993).

### BRIEF SUMMARY OF THE INVENTION

One aspect of the invention provides a sterile pharmaceutical formulation suitable for parenteral administration comprising: (a) a recombinant protein; (b) sodium acetate; (c) arginine hydrochloride; (d) a cryoprotecant; and (e) a surfactant.

Another aspect of the invention provides a sterile pharmaceutical formulation suitable for parenteral administration comprising: (a) an antibody selected from an anti-HER2, anti-VEGF, anti-CD20, anti-TNFa, anti-EGFR, anti-VEGF Fab, anti-IgE, anti-α4 integrin, anti-TNFα, anti-CD25, anti-IL6, anti-05, anti-CD52, anti-CTLA4, anti-RANKL, anti-BLyS, anti-glyco IIb/IIIa Fab, anti-IL12&23, anti-interleukin-1b and an anti-CD3R antibody; (b) sodium acetate; (c) arginine hydrochloride; (d) a cryoporotectant; and (e) a surfactant, wherein said formulation has a pH of about 5.2, and wherein said formulation contains a lower percent of high molecular weight species than a corresponding formulation lacking arginine hydrochloride following storage of each formulation at 40° C. for 4 weeks.

### BRIEF DESCRIPTION OF THE DRAWINGS

FIG. 1 illustrates HPLC chromatograms for three TX16 formulations and shows the effects of different buffers on the relative levels of high molecular weight species (HMWS).

FIG. 2 illustrates high molecular weight species formation over time for three sets of formulations, and shows the effects of L-arginine HCl on HWMS formation over time.

FIG. 3 illustrates SEC chromatograms showing the effect of L-arginine HCl on levels of dissociable HMWS.

FIG. 4 illustrates the effects of L-arginine HCl on the formation of dissociable HMWS of various formulations at 0° C. and 40° C.

FIG. 5 illustrates CEX chromatograms showing the effects of L-arginine HCl on the chemical stability of a TX16 formulation at 40° C.

FIG. 6 illustrates the effect of L-arginine HCl on the formation of TX16 acidic variants over time in formulations having different buffers.

FIG. 7 the effect of L-arginine HCl on the formation of HMWS in various TX16 formulations upon being subjected to physical stress of freeze thaw cycles.

FIG. 8 illustrates the effect of L-arginine HCl on the HMWS concentration of various TX16 formulations having either sodium acetate or sodium phosphate buffer.

FIG. 9 illustrates the effect of polysorbate 20 on the concentration of HMWS in TX16 formulations exposed to physical stress by agitation for up to 48 hours.

FIG. 10 illustrates the effect of polysorbate 20 on the turbidity of TX16 formulations exposed to physical stress by agitation for 24 hours.

### DETAILED DESCRIPTION OF THE INVENTION

### Definitions

As used herein, the following definitions shall apply unless otherwise indicated.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used herein, a “sterile” formulation is aseptic or free or essentially free from living microorganisms and their spores.

As used herein, a “stable” formulation is one in which the protein therein essentially retains its physical stability, chemical stability and biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993), for example. Stability can be measured at a selected temperature for a selected time period. In certain embodiments, the formulation is stable at about 40° C. for 1 week, 2 weeks, 3 week or 4 weeks. In addition, in certain embodiments the formulation is stable following freezing (e.g., −20° C. to −70° C.) and thawing of the formulation. For example, in certain embodiments the formulation is stable upon exposure to anywhere from one to five cycles of freezing and thawing. Stability can be evaluated qualitatively and/or quantitatively in a variety of different ways, including evaluation of aggregate formation (for example using size exclusion chromatography, by measuring turbidity, and/or by visual inspection); by assessing charge heterogeneity using cation exchange chromatography, image capillary isoelectric focusing (icIEF) or capillary zone electrophoresis; amino-terminal or carboxy-terminal sequence analysis; mass spectrometric analysis; SDS-PAGE analysis to compare reduced and intact antibody; peptide map (for example tryptic or LYS-C) analysis; evaluating biological activity or antigen binding function of the antibody; etc. Instability may involve any one or more of: aggregation, deamidation (e.g. Asn deamidation), oxidation (e.g. Met oxidation), isomerization (e.g. Asp isomeriation), clipping/hydrolysis/fragmentation (e.g. hinge region fragmentation), succinimide formation, unpaired cysteine(s), N-terminal extension, C-terminal processing, glycosylation differences, etc.

A protein exhibits physical stability in a pharmaceutical formulation if it shows little to no aggregation, precipitation and/or denaturation upon visual examination of color and/or clarity, or as measured by UV light scattering or by size exclusion chromatography.

A protein exhibits chemical stability in a pharmaceutical formulation, if the chemical stability at a given time is such that the protein is considered to still retain its biological activity as defined below. Chemical stability can be assessed by detecting and quantifying chemically altered forms of the protein. Chemical alteration may involve size modification (e.g. clipping) which can be evaluated using size exclusion chromatography, CE-SDS (non-reduced) and/or SDS-PAGE and/or matrix-assisted laser desorption ionization/time-of-flight mass spectrometry (MALDI/TOF MS), for example. Other types of chemical alteration include charge alteration (e.g. occurring as a result of deamidation) which can be evaluated by ion-exchange chromatography or icIEF, for example.

An antibody retains its biological activity in a pharmaceutical formulation, if the biological activity of the antibody at a given time is within about 10% (within the errors of the assay) of the biological activity exhibited at the time the pharmaceutical formulation was prepared as determined in an antigen binding assay, for example.

As used herein, “biological activity” of a monoclonal antibody refers to the ability of the antibody to bind to antigen. It can further include antibody binding to antigen and resulting in a measurable biological response which can be measured in vitro or in vivo. Such activity may be antagonistic or agonistic.

By “isotonic” is meant that the formulation of interest has essentially the same osmotic pressure as human blood. Isotonic formulations will generally have an osmotic pressure from about 240 to 350 mOsm. Isotonicity can be measured using a vapor pressure or ice-freezing type osmometer, for example.

As used herein, the term “buffered solution” refers to a solution that resists changes in pH by the action of its acid-base conjugate components. The buffer of this invention preferably has a pH in the range from about 3.5 to about 6.5, typically from about 4.6 to about 5.8, for example from 4.8 to 5.6 or 5.0 to 5.4. In one embodiment the buffer has a pH of about 5.2. Examples of buffers that will control the pH of the formulations of the present invention in this range include acetate, succinate, succinate, gluconate, histidine, citrate and glycylglycine.

As used herein, a “surfactant” refers to a surface-active agent, typically a nonionic surfactant. Examples of surfactants suitable for the formulations of this invention include polysorbate (for example, polysorbate 20 and, polysorbate 80); poloxamer (e.g. poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; polyethyl glycol, polypropyl glycol, and copolymers of ethylene and propylene glycol (e.g. Pluronics, PF68 etc). In one embodiment, the surfactant herein is polysorbate 20.

In a pharmacological sense, in the context of the invention, a “therapeutically effective amount” of an antibody refers to an amount effective in the prevention or treatment of a disorder for the treatment of which the antibody is effective. A “disorder” is any condition that would benefit from treatment with the antibody. This includes chronic and acute disorders or diseases including those pathological conditions which predispose the mammal to the disorder in question.

As used herein, a “preservative” is a compound that inhibits bacterial growth. Examples of potential preservatives in the formulations of the present invention include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. In one embodiment, the preservative herein is benzyl alcohol.

The term “recombinant protein” refers to polymeric amino acids molecules manufactured by recombinant DNA techniques.

The term “cryoprotectant” refers to a substance, such as a chemical compound or molecule, that protects a protein from damage resulting from being frozen. For example, cryoprotectants may be used to protect a recombinant protein from denaturization during exposure to freezing and thawing cycles.

The term “VEGF” or “VEGF-A” as used herein refers to the 165-amino acid human vascular endothelial cell growth factor and related 121-, 189-, and 206-amino acid human vascular endothelial cell growth factors, as described by Leung et al. (1989) Science 246:1306, and Houck et al. (1991) Mol. Endocrin, 5:1806, together with the naturally occurring allelic and processed forms thereof. The term “VEGF” also refers to VEGFs from non-human species such as mouse, rat or primate. Sometimes the VEGF from a specific species are indicated by terms such as hVEGF for human VEGF, mVEGF for murine VEGF, and etc. The term “VEGF” is also used to refer to truncated forms of the polypeptide comprising amino acids 8 to 109 or 1 to 109 of the 165-amino acid human vascular endothelial cell growth factor. Reference to any such forms of VEGF may be identified in the present application, e.g., by “VEGF (8-109),” “VEGF (1-109)” or “VEGF.sub.165.” The amino acid positions for a “truncated” native VEGF are numbered as indicated in the native VEGF sequence. For example, amino acid position 17 (methionine) in truncated native VEGF is also position 17 (methionine) in native VEGF. The truncated native VEGF has binding affinity for the KDR and Flt-1 receptors comparable to native VEGF.

A “VEGF antagonist” or “VEGF-specific antagonist” refers to a molecule capable of binding to VEGF, reducing VEGF expression levels, or neutralizing, blocking, inhibiting, abrogating, reducing, or interfering with VEGF biological activities, including, but not limited to, VEGF binding to one or more VEGF receptors and VEGF mediated angiogenesis and endothelial cell survival or proliferation. Included as VEGF-specific antagonists useful in the methods of the invention are polypeptides that specifically bind to VEGF, anti-VEGF antibodies and antigen-binding fragments thereof, receptor molecules and derivatives which bind specifically to VEGF thereby sequestering its binding to one or more receptors, fusions proteins (e.g., VEGF-Trap (Regeneron)), and VEGF.sub.121-gelonin (Peregrine). VEGF-specific antagonists also include antagonist variants of VEGF polypeptides, antisense nucleobase oligomers directed to VEGF, small RNA molecules directed to VEGF, RNA aptamers, peptibodies, and ribozymes against VEGF. VEGF-specific antagonists also include nonpeptide small molecules that bind to VEGF and are capable of blocking, inhibiting, abrogating, reducing, or interfering with VEGF biological activities. Thus, the term “VEGF activities” specifically includes VEGF mediated biological activities of VEGF. In certain embodiments, the VEGF antagonist reduces or inhibits, by at least 10%, 20%, 30%, 40%, 50%, 60%, 70%, 80%, 90% or more, the expression level or biological activity of VEGF.

An “anti-VEGF antibody” is an antibody that binds to VEGF with sufficient affinity and specificity. In certain embodiments, the antibody selected will normally have a sufficiently binding affinity for VEGF, for example, the antibody may bind hVEGF with a K.sub.d value of between 100 nM-1 pM. Antibody affinities may be determined by a surface plasmon resonance based assay (such as the BIAcore assay as described in PCT Application Publication No. WO2005/012359); enzyme-linked immunoabsorbent assay (ELISA); and competition assays (e.g. RIA's), for example.

In certain embodiment, the anti-VEGF antibody can be used as a therapeutic agent in targeting and interfering with diseases or conditions wherein the VEGF activity is involved. Also, the antibody may be subjected to other biological activity assays, e.g., in order to evaluate its effectiveness as a therapeutic. Such assays are known in the art and depend on the target antigen and intended use for the antibody. Examples include the HUVEC inhibition assay; tumor cell growth inhibition assays (as described in WO 89/06692, for example); antibody-dependent cellular cytotoxicity (ADCC) and complement-mediated cytotoxicity (CDC) assays (U.S. Pat. No. 5,500,362); and agonistic activity or hematopoiesis assays (see WO 95/27062). An anti-VEGF antibody will usually not bind to other VEGF homologues such as VEGF-B or VEGF-C, nor other growth factors such as P1GF, PDGF or bFGF. In one embodiment, anti-VEGF antibody is a monoclonal antibody that binds to the same epitope as the monoclonal anti-VEGF antibody A4.6.1 produced by hybridoma ATCC HB 10709. In another embodiment, the anti-VEGF antibody is a recombinant humanized anti-VEGF monoclonal antibody generated according to Presta et al. (1997) Cancer Res. 57:4593-4599, including but not limited to the antibody known as bevacizumab. Bevacizumab, also known as AVASTIN®, and other humanized anti-VEGF antibodies are described in U.S. Pat. No. 6,884,879, the entire contents of which are expressly incorporated by reference herein.

The term “antibody” is used herein in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

An “isolated” antibody is one which has been identified and separated and/or recovered from a component of its natural environment. Contaminant components of its natural environment are materials which would interfere with research, diagnostic or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. Isolated antibody includes the antibody in situ within recombinant cells since at least one component of the antibody's natural environment will not be present. Ordinarily, however, isolated antibody will be prepared by at least one purification step.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V.sub.H) followed by a number of constant domains. Each light chain has a variable domain at one end (V.sub.L) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The term “constant domain” refers to the portion of an immunoglobulin molecule having a more conserved amino acid sequence relative to the other portion of the immunoglobulin, the variable domain, which contains the antigen binding site. The constant domain contains the C.sub.H1, C.sub.H2 and C.sub.H3 domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The terms “full length antibody,” “intact antibody” and “whole antibody” are used herein interchangeably to refer to an antibody in its substantially intact form, not antibody fragments as defined below. The terms particularly refer to an antibody with heavy chains that contain an Fc region.

A “naked antibody” for the purposes herein is an antibody that is not conjugated to a cytotoxic moiety or radiolabel.

“Antibody fragments” comprise a portion of an intact antibody, preferably comprising the antigen binding region thereof. Examples of antibody fragments include Fab, Fab′, F(ab′).sub.2, and Fv fragments; diabodies; linear antibodies; single-chain antibody molecules; and multispecific antibodies formed from antibody fragments.

The term “monoclonal antibody” as used herein refers to an antibody obtained from a population of substantially homogeneous antibodies, e.g., the individual antibodies comprising the population are identical except for possible variants that may arise during production of the monoclonal antibody, such variants generally being present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATTZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR of the recipient are replaced by residues from a HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr Op. Biotech. 5:428-433 (1994); and U.S. Pat. Nos. 6,982,321 and 7,087,409.

The antibody which is formulated is preferably essentially pure and desirably essentially homogeneous (e.g., free from contaminating proteins etc). “Essentially pure” antibody means a composition comprising at least about 90% by weight of the antibody, based on total weight of the composition, preferably at least about 95% by weight. “Essentially homogeneous” antibody means a composition comprising at least about 99% by weight of antibody, based on total weight of the composition.

It is understood that the above definitions are not intended to include impermissible substitution patterns (e.g., methyl substituted with 5 fluoro groups). Such impermissible substitution patterns are easily recognized by a person having ordinary skill in the art.

Antibody Formulations

In one aspect, the invention provides a sterile pharmaceutical formulation suitable for parenteral administration comprising: (a) a recombinant protein; (b) sodium acetate; (c) arginine hydrochloride; (d) a cryoprotecant; and (e) a surfactant.

Another aspect of the invention provides a sterile pharmaceutical formulation suitable for parenteral administration comprising: (a) an antibody selected from an anti-HER2, anti-VEGF, anti-CD20, anti-TNFa, anti-EGFR, anti-VEGF Fab, anti-IgE, anti-α4 integrin, anti-TNFα, anti-CD25, anti-IL6, anti-CS, anti-CD52, anti-CTLA4, anti-RANKL, anti-BLyS, anti-glyco IIb/IIIa Fab, anti-IL12&23, anti-interleukin-1b and an anti-CD3R antibody; (b) sodium acetate; (c) arginine hydrochloride; (d) a cryoporotectant; and (e) a surfactant, wherein said formulation has a pH of about 5.2, and wherein said formulation contains a lower percent of high molecular weight species than a corresponding formulation lacking arginine hydrochloride following storage of each formulation at 40° C. for 4 weeks.

In one embodiment the recombinant protein is selected from anti-HER2, anti-VEGF, anti-CD20, anti-TNFa, anti-EGFR, anti-VEGF Fab, anti-IgE, anti-α4 integrin, anti-TNFα, Anti-CD25, Anti-IL6, Anti-CS, Anti-CD52, Anti-CTLA4, Anti-RANKL, Anti-BLyS, Anti-glyco IIb/IIIa Fab, Anti-IL12&23, Anti-interleukin-1b, Anti-CD3R, interferon beta-1a, GCSF, PEG-GCSF, CTLA4-Fc, Anti-CD20 yttrium 90, Anti-CD20 iodine 131, TNFaR-Fc and anti-F Protein.

In certain embodiments the recombinant protein is an antibody. In certain embodiments where the recombinant protein is an antibody, the antibody is an anti-HER2, anti-VEGF, anti-CD20, anti-TNFa, anti-EGFR, anti-VEGF Fab, anti-IgE, anti-α4 integrin, anti-TNFα, anti-CD25, anti-IL6, anti-CS, anti-CD52, anti-CTLA4, anti-RANKL, anti-BLyS, anti-glyco IIb/IIIa Fab, anti-IL12&23, anti-interleukin-1b or an anti-CD3R antibody. In another embodiment the antibody is an anti-HER2, anti-VEGF, anti-CD20, anti-TNFa, anti-VEGF Fab, anti-EGFR or anti-IgE antibody. In still another embodiment the antibody is an anti-VEGF antibody. In one such embodiment the antibody is TX16.

The monoclonal antibodies to be used in accordance with the present invention may be made by the hybridoma method first described by Kohler et al., Nature, 256:495 (1975), or may be made by recombinant DNA methods (U.S. Pat. No. 4,816,567). The “monoclonal antibody” may also be isolated from phase antibody libraries using the techniques described in Clackson et al., Nature, 352:624-628 (1991) and Marks et al., J. Mol. Biol., 222:581-597 (1991).

The concentration of recombinant protein in the formulation may be from 10 mg/mL to 200 mg/mL. In certain embodiments the concentration of recombinant protein is from 20 mg/mL to 80 mg/mL. In one embodiment the concentration of recombinant protein in the formulation is about 20 mg/mL, about 25 mg/mL, about 30 mg/mL, about 35 mg/mL, about 40 mg/mL, about 45 mg/mL or about 50 mg/mL. In one such embodiment the concentration of recombinant protein in the formulation is about 25 mg/mL.

The concentration of sodium acetate in the formulation may be from 10 mM to 250 mM. In certain embodiments the concentration of sodium acetate is from 10 mM to 50 mM. In one embodiment the concentration of sodium acetate in the formulation is about 15 mM, about 20 mM, about 25 mM, about 30 mg/mL or about 40 mM. In one such embodiment the concentration of sodium acetate in the formulation is about 20 mM.

The concentration of arginine hydrochloride in the formulation may be from 20 mM to 250 mM. In certain embodiments the concentration of sodium acetate is from 50 mM to 150 mM. In one embodiment the concentration of sodium acetate in the formulation is about 80 mM, about 90 mM, about 100 mM, about 110 mM or about 120 mM. In one such embodiment the concentration of arginine hydrochloride in the formulation is about 100 mM.

The formulations of the present invention include a cryoprotectant. In certain embodiments, the cryoprotectant is a monosaccharide, a disaccharide or a sugar alcohol. In one embodiment the cryoprotectant is glucose, fructose, sucrose, trehalose, lactose, maltose, lactulose, sorbitol or mannitol. In one such embodiment the cryoprotectant is sucrose or trehalose. In one embodiment, the cryoprotectant is sucrose.

The concentration of cryoprotectant in the formulation may be from 10 mM to 250 mM. In certain embodiments the concentration of cryoprotectant is from 100 mM to 150 mM. In one embodiment the concentration of cryoprotectant in the formulation is about 100 mM, about 110 mM, about 120 mM, about 130 mg/mL or about 140 mM. In one such embodiment the concentration of sodium acetate in the formulation is about 120 mM. For example, in one embodiment the cryoprotectant is sucrose present at a concentration of about 120 mM.

The formulations of the present invention also include a surfactant. In certain embodiments the surfactant is a polysorbate or a poloxamer. In one embodiment the surfactant is polysorbate 20, polysorbae 80 or poloxamer 188. For example, in one embodiment the surfactant is polysorbate 20.

The concentration of surfactant in the formulation may be from 0.005% by weight to 0.5% by weight. In certain embodiments the concentration of surfactant is from 0.01% by weight to 0.1% by weight. In one embodiment the concentration of surfactant in the formulation is from 0.01% by weight to 0.1% by weight. For example, in one embodiment the surfactant is polysorbate 20 present at a concentration of about 0.04% by weight.

The formulations of the present invention have a pH in the range of 3.5 to 6.5. In certain embodiments, the pH of the formulation is from 4.6 to 5.8. In certain embodiments, the pH of the formulation is from 4.8 to 5.6. In certain embodiments, the pH of the formulation is from 5.0 to 5.4. For example, in one embodiment the formulation has a pH of about 5.2.

### EXAMPLES

### Analytical Methods

### Color, Appearance and Clarity

The color, appearance, and clarity of the samples were determined by visual inspection of vials against a white and black background under white fluorescence light at room temperature.

UV Concentration Measurements

The liquid product aliquot was first diluted with formulation buffer so that the Aₘₐₓ near 280 nm is within 0.5-1.0 absorbance unit. The UV absorbance of the diluted samples was measured in a quartz cuvette with 1 cm path length on an HP 8453 spectrophotometer. Absorbance was measured at 280 nm and 320 nm. The absorbance from 320 nm is used to correct background light scattering due to larger aggregates, bubbles and particles. The measurements were blanked against the formulation buffer. The protein concentration was determined using the absorptivity of 1.70 (mg/mL)⁻¹ cm⁻¹.

Turbidity (UV-Vis 340-360 nm)

The liquid product aliquot was measured undiluted at absorbance from 340-360 nm. The UV-Vis absorbance of the samples was measured in a quartz cuvette with 1 cm path length on an HP 8453 spectrophotometer. The absorbance from 340-360 nm is used to measure light scattering due to larger aggregates, bubbles and particles/insoluble aggregates. The measurements were blanked against the formulation buffer.

PH Measurements

The pH was measured at room temperature using a Metler Toledo MultiSeven™ pH meter. The probe used was Metler Toledo InLab® pH combination electrode (InLab® Expert Pro-ISM-IP67). Standard solutions of pH 4.01 and pH 7.00 (BDH®) were used for calibration of the pH meter.

Ion-Exchange Chromatography

Cation exchange chromatography was employed to measure changes in charge variants. This assay utilizes a Dionex ProPac WCX-10 HT column on an Dionex Ultimate 3000 system. Samples were diluted to 1 mg/mL with the mobile phase A containing 2.4 mM Tris.HCl, 1.5 mM Imidazole, 11.6 mM Piperazine.2HCl.H2O, pH 6.0. 50 μL of diluted samples were then loaded on the column that was kept at 4° C. temperature. The peaks were eluted with a shallow pH gradient using mobile B containing 2.4 mM Tris.HCl, 1.5 mM Imidazole, 11.6 mM Piperazine.2HCl.H2O, pH 9.5. The eluent was monitored at 280 nm. The data were analyzed using a Dionex software (Chromeleon version 7.0).

Size Exclusion Chromatography

Size exclusion chromatography was used to quantitate aggregates and fragments. This assay utilizes a TSK G3000 SWXL, 7.8×300 mm column and runs on Dionex Ultimate 3000 system. Samples were diluted to 10 mg/mL with the mobile phase and injection volume was 20 uL. The mobile phase was 250 mM K₂HPO₄, 200 mM KCl at pH 7.0 and the protein was eluted with an isocratic gradient at 0.5 mL/min for 30 minutes. The eluent absorbance was monitored at 280 nm. Integration was done using Dionex software (Chromeleon version 7.0).

### Example 1

### TX16 Liquid Formulation Preparation

TX16 was buffer exchanged into various buffer by using slide-A-Lyzer cassettes. The final formulation containing 25 mg/mL of TX16 is listed in Table 1. The formulations were sterile filtered with 0.22 μm filter (Steriflip, MilliPore) and aseptically filled into presterilized USP Type 1 glass vials, stoppered with gray butyl Florotec™ coated stopper and capped with aluminum flip-top cap. The formulations were evaluated using SEC and CEX HPLC methods for molar mass distribution and charge variants profile, respectively.

TABLE 1
TX16 Formulations

| Formulation No. | Formulation composition |
|---|---|
| 1 | 50 mM Sodium phosphate, 6% trehalose, 0.04% |
| | polysorbate 20, pH 6.2 |
| 2 | 20 mM Histidine-Hydrochloride, 120 mM sucrose, |
| | 0.04% polysorbate 20, pH 6.2 |
| 3 | 20 mM Sodium Acetate, 100 mM L-arginine |
| | hydrochloride, 120 mM sucrose, |
| | 0.04% polysorbate 20, pH 5.2 |

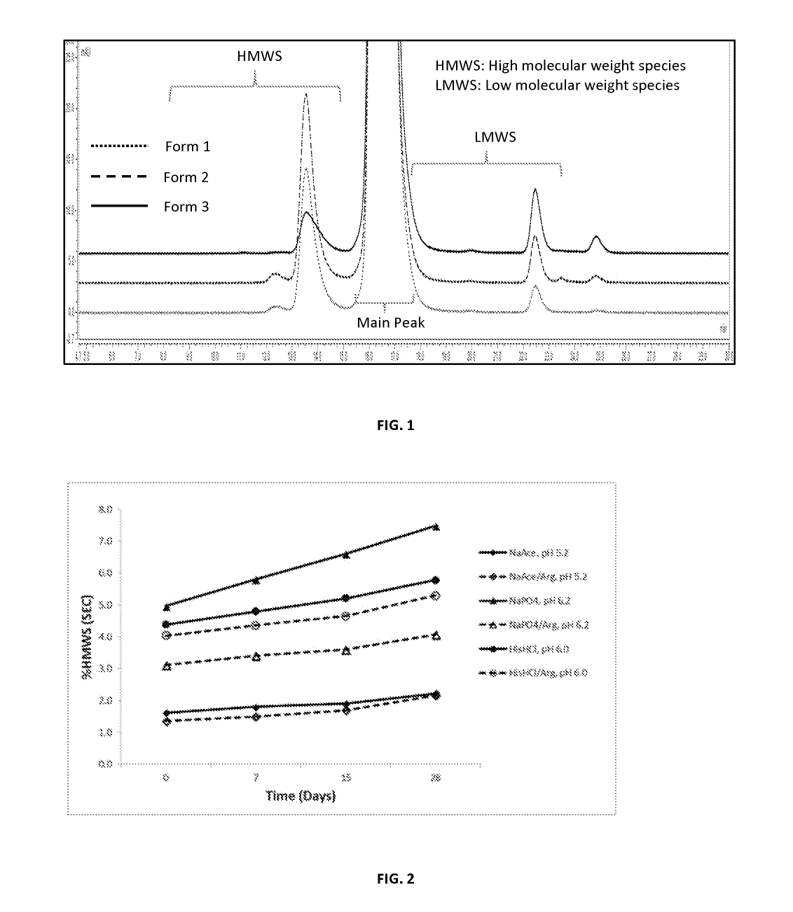

The combination of lower pH (pH 5.2) in sodium acetate buffer, sucrose, polysorbate 20, and addition of L-arginine hydrochloride produced a stable formulation that yielded the lowest amount of high molecular weight species (HMWS) formation. Formulation 1 (Sodium phosphate buffer) yield 4.7% HMWS, Formulation 2 (histidine hydrochloride buffer) yield 5.9% HMWS, and Formulation 3 (sodium acetate buffer) yield 1.4% HMWS, an amount that is significantly lower than formulation 1 and 2. See FIG. 1.

### Example 2

### TX16 Liquid Formulation Preparation

TX16 was buffer exchanged into various buffer by using slide-A-Lyzer cassettes. The final formulation containing 25 mg/mL of TX16 is listed in Table 2. The formulations were sterile filtered with 0.22 μm filter (Steriflip, MilliPore) and aseptically filled into presterilized USP Type 1 glass vials, stoppered with gray butyl Florotec™ coated stopper and capped with aluminum flip-top cap.

TABLE 2
TX16 Formulations

| Formulation | |
|---|---|
| No. | Formulation composition |
| 1 | 20 mM Sodium Acetate, 120 mM sucrose, |
| | 0.04% polysorbate 20 |
| 2 | 20 mM Sodium Acetate, 100 mM L-arginine hydrochloride, |
| | 120 mM sucrose, 0.04% polysorbate 20 |
| 3 | 20 mM Histidine-Hydrochloride, 120 mM sucrose, |
| | 0.04% polysorbate 20 |
| 4 | 20 mM Histidine-Hydrochloride, 100 mM L-arginine |
| | hydrochloride, 120 mM sucrose, 0.04% polysorbate 20 |
| 5 | 20 mM Sodium Phosphate, 120 mM sucrose, |
| | 0.04% polysorbate 20 |
| 6 | 20 mM Sodium Phosphate, 100 mM L-arginine |
| | hydrochloride, 120 mM sucrose, 0.04% polysorbate 20 |

The formulations were evaluated using SEC and CEX HPLC methods for molar mass distribution and charge variants profile, respectively. Formulations containing L-arginine hydrochloride showed reduction in high molecular weight species (HMWS) formation at T=0 as well as over time (Days) at an elevated storage temperature of 40° C. See FIG. 2.

### Example 3

### Effect of L-arginine HCl on Dissociable HMWS

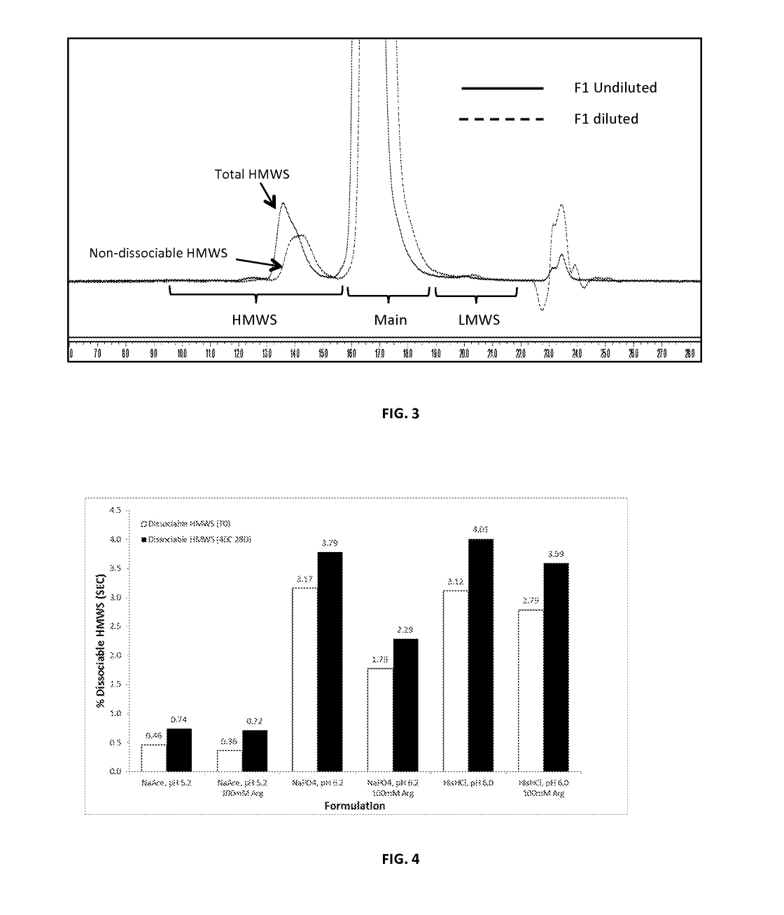

Part of TX16 high molecular weight species (HMWS) can be dissociated upon dilution to 0.5 mg/mL followed by incubation at 30° C. for 24 hours. The dissociable HMWS is equal to the subtraction of non-dissociable aggregate (tested when diluted to 0.5 mg/mL) from total HMWS (tested undiluted). See FIG. 3.

Formulations 1-6 (Table 2) were evaluated for dissociable HMWS formation. TX16 formulated in sodium acetate (pH 5.2), sucrose, L-arginine HCl, and PS20 yielded the least dissociable HMWS at T=0 and storage at an elevated temperature of 40° C. Overall, addition of arginine HCl reduced the amount of dissociable HMWS as shown in the samples exposed to thermal stress at 40° C. for 28 days. See FIG. 4.

### Example 4

### Effect of Chemical Stability with Addition of L-Arginine HCl

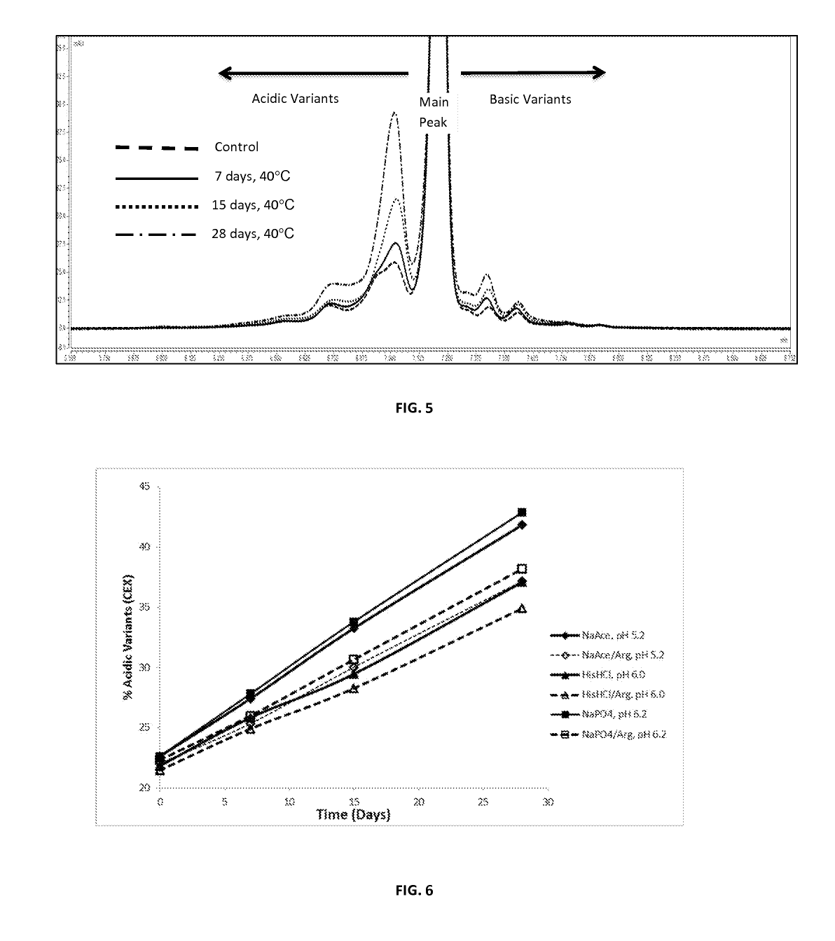

TX16 charge variant is evaluated by a pH gradient elution HPLC method. As shown in FIG. 5, acidic variants formation is the major degradation pathway for TX16 evaluated under accelerated condition at an elevated temperature of 40° C. over time.

TX16 formulations with and without L-arginine HCl and degraded under accelerated conditions at 40° C. were evaluated with respect to acidic variants formation over time. FIG. 6 shows that TX16 formulations containing L-arginine HCl were more stable, forming lower amount of acidic variants over time.

### Example 5

### Effect of TX16 Formulations Against Physical Stress in Freeze Thaw Cycles

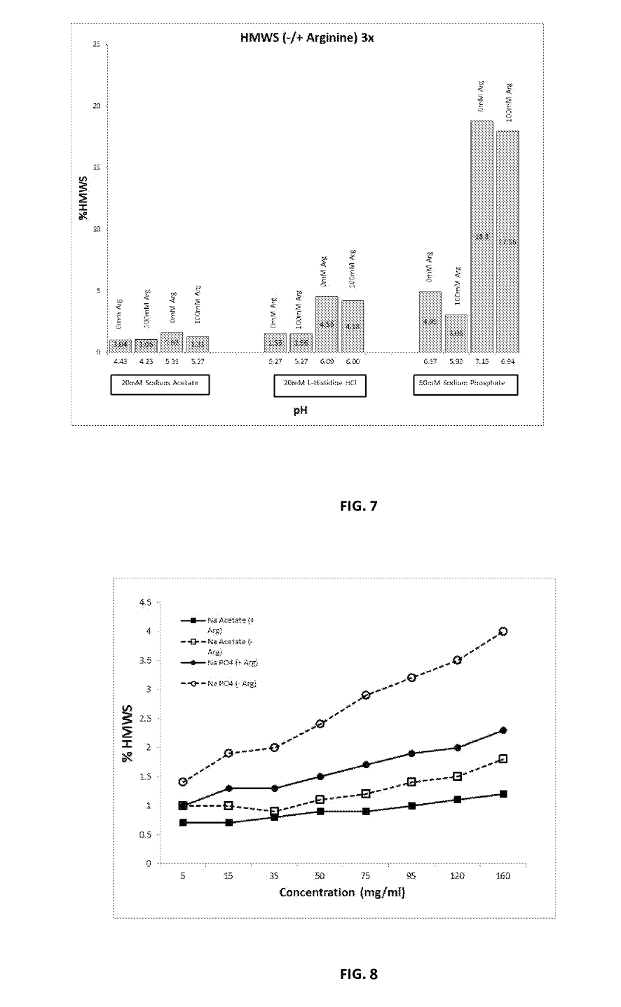

TX16 formulations were formulated with or without L-arginine HCl were subjected to physical stress by repeating freezing and thawing cycle for at least 3 cycles. All formulations in this study contained equal amount of sucrose as a primary cyroprotectant. FIG. 7 shows that formulations containing L-arginine HCl had lower amount of HMWS after three freeze thaw cycles in sodium acetate, histidine HCl and sodium phosphate buffer system. The results showed addition of L-arginine HCL further improvement of physical stability during freezing and thawing process in the presence of the existing cyroprotectant.

### Example 6

### Effect of L-Arginine HCl in on HMWS Formation in Wide Range of Protein Concentration (5-160 mg/mL)

TX16 Liquid Formulation Preparation

TX16 was buffer exchanged into various bufferS by using slide-A-Lyzer cassettes. The resultING protein solution was further concentrated using Amicon Ultra-15 (Millipore, part no. UFC901024). A 0.5 mL fraction of sample was pulled upon concentration increase to 20-30 mg/mL. All samples were tested for protein concentration (UV-Vis) and HMWS formation using SEC method.

FIG. 8 shows that TX16 formulations having 100 mM of L-arginine HCl and either sodium acetate buffer or sodium phosphate buffer had lower amounts of HMWS formation at a wide range of protein concentrations (5-160 mg/mL) compared to those formulated in the same buffer without addition of L-arginine HCl.

### Example 7

### TX16 Formulated in Sodium Acetate with L-Arginine and Polysorbate 20 (PS20) is Stable Against Agitation Stress

TX16 Liquid Formulation Preparation

TX16 was formulated in sodium acetate with and without L-arginine-HCl and with and without PS20 as listed on Table 3.

TABLE 3
Formulation Composition for Agitation Stress Study

| | | % Polysorbate |
|---|---|---|
| Formulation | | 20 |
| No. | Formulation Composition | (% wt/V) |
| 1 | 25 mg/mL TX16 | 0% |
| | 20 mM sodium acetate, pH 5.2 | |
| | 100 mM Arginine - HCl | |
| | 120 mM Sucrose | |
| 2 | 25 mg/mL TX16 | 0.04% |
| | 20 mM sodium acetate, pH 5.2 | |
| | 100 mM Arginine - HCl | |
| | 120 mM Sucrose | |

These formulations were filled into 2-mL USP type 1 glass vials with 0.8 mL sample volume and agitated for 24 hours using a bench top shaker at 100 rpm and 8 cm away from the center of the shaker. Both control and stressed samples were tested for % HMWS to assess soluble aggregates formation and turbidity for insoluble aggregates formation.

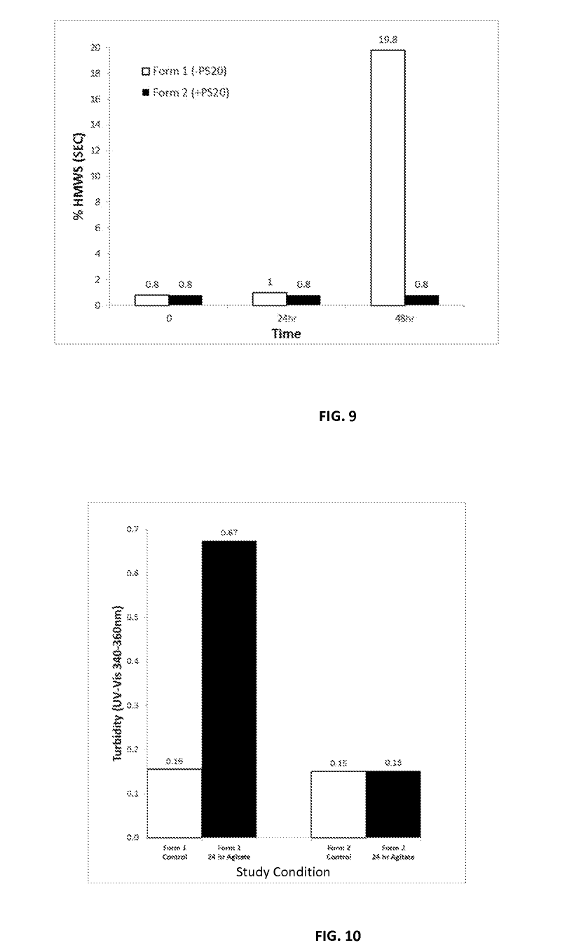

FIG. 9 shows that the formulation containing polysorbate 20 (Formulation No. 2) is stable against agitation stress by showing no increase in % HMWS after 48 hours of vigorous agitation. In contrast, formulation 1 which contain no polysorbate 20 showed significant increase in % HMWS from 0.8% to 19.8% when agitated for 48 hours. Formulation 2 containing PS20 also showed no increase in turbidity (UV-Vis 340-360 nm), which indicates no increase in insoluble aggregates formation. In contrast, formulation without PS20 (Formulation 1) showed a significant increase in turbidity measurement after 24 hours of agitation, demonstrating insoluble aggregates formation without presence of non-ionic surfactant PS20 (FIG. 10). These results demonstrated that polysorbate 20 is essential to stabilize TX16 against physical stress.

Overall, TX16 formulations containing sodium acetate buffer at pH 5.2, 120 mM Sucrose, 100 mM L-arginine HCl, and 0.04% polysorbate 20 exhibit excellent physical and chemical stability.

While particular embodiments of the present invention have been shown and described herein for purposes of illustration, it will be understood, of course, that the invention is not limited thereto since modifications may be made by persons skilled in the art, particularly in light of the foregoing teachings, without deviating from the spirit and scope of the invention. Accordingly, the invention is not limited except as by the appended claims.

All of the U.S. patents, U.S. patent application publications, U.S. patent applications, foreign patents, foreign patent applications and non-patent publications referred to in this specification are incorporated herein by reference, in their entirety to the extent not inconsistent with the present description.

We claim:

1. A sterile pharmaceutical formulation suitable for parenteral administration comprising: (a) a recombinant protein; (b) sodium acetate; (c) arginine hydrochloride; (d) a cryoporotectant; and (e) a surfactant.

2. A formulation according to claim 1, wherein said recombinant protein is selected from anti-HER2, anti-VEGF, anti-CD20, anti-TNFa, anti-EGFR, anti-VEGF Fab, anti-IgE, anti-α4 integrin, anti-TNFα, anti-CD25, anti-IL6, anti-05, Anti-CD52, anti-CTLA4, anti-RANKL, anti-BLyS, anti-glyco IIb/IIIa Fab, anti-IL12&23, anti-interleukin-1b, anti-CD3R, interferon beta-1a, GCSF, PEG-GCSF, CTLA4-Fc, anti-CD20 yttrium 90, anti-CD20 iodine 131, TNFaR-Fc and anti-F Protein.

3. (canceled)

4. A formulation according to claim 3, wherein said antibody is an anti-HER2, anti-VEGF, anti-CD20, anti-TNFa, anti-VEGF Fab, anti-EGFR or anti-IgE antibody.

5. A formulation according to claim 4, wherein the concentration of said antibody is from 10 mg/mL to 200 mg/mL, or from 20 mg/mL to 80 mg/mL, or from 20 mg/mL to 30 mg/mL.

6-8. (canceled)

9. A formulation according to claim 1, wherein the concentration of sodium acetate is from 10 mM to 250 Mm, or from 10 mM to 50 mM.

10-11. (canceled)

12. A formulation according to claim 1, wherein the concentration of arginine hydrochloride is from 20 mM to 250 mM, or from 50 mM to 150 mM.

13-14. (canceled)

15. A formulation according to claim 1, wherein said cryoprotectant is a monosaccharide, a disaccharide or a sugar alcohol.

16. A formulation according to claim 15, wherein said cryoprotectant is glucose, fructose, sucrose, trehalose, lactose, maltose, lactulose, sorbitol or mannitol.

17-18. (canceled)

19. A formulation according to claim 16, wherein the concentration of sucrose is from 10 mM to 250 mM, or from 100 mM to 150 mM.

20-21. (canceled)

22. A formulation according to claim 1, wherein said surfactant is a nonionic polymeric surfactant.

23-25. (canceled)

26. A formulation according to claim 22, wherein said surfactant is polysorbate 20 and wherein the concentration of polysorbate 20 is from 0.005% by weight to 0.5% by weight, or from 0.01% by weight to 0.1% by weight, or from 0.02% by weight to 0.05% by weight.

27-29. (canceled)

30. A formulation according to claim 1, having a pH from 3.5 to 6.5.

31-35. (canceled)

36. A sterile antibody formulation suitable for parenteral administration comprising: (a) an antibody selected from an anti-HER2, anti-VEGF, anti-CD20, anti-TNFa, anti-EGFR, anti-VEGF Fab, anti-IgE, anti-α4 integrin, anti-TNFα, anti-CD25, anti-IL6, anti-CS, anti-CD52, anti-CTLA4, anti-RANKL, anti-BLyS, anti-glyco IIb/IIIa Fab, anti-IL12&23, anti-interleukin-1b and an anti-CD3R antibody; (b) sodium acetate; (c) arginine hydrochloride; (d) a cryoporotectant; and (e) a surfactant, wherein said formulation has a pH of about 5.2, and wherein said formulation contains a lower percent of high molecular weight species than a corresponding formulation lacking arginine hydrochloride following storage of each formulation at 40° C. for 4 weeks.

37. A formulation according to claim 36, wherein said antibody is an anti-HER2, anti-VEGF, anti-CD20, anti-TNFa, anti-VEGF Fab, anti-EGFR or anti-IgE antibody.

38. A formulation according to claim 37, wherein the concentration of said antibody is from 20 mg/mL to 30 mg/mL.

39. A formulation according to claim 37, wherein the concentration of said antibody is about 25 mg/mL.

40. A formulation according to claim 37, wherein the concentration of sodium acetate is from 10 mM to 50 mM.

41. (canceled)

42. A formulation according to claim 36, wherein the concentration of arginine hydrochloride is from 50 mM to 150 mM.

43. (canceled)

44. A formulation according to claim 36, wherein said cryoprotectant is sucrose, and wherein the concentration of sucrose is from 100 mM to 150 mM.

45-46. (canceled)

47. A formulation according to claim 36, wherein said surfactant is polysorbate 20, and wherein the concentration of polysorbate 20 is from 0.01% by weight to 0.1% by weight.

48-50. (canceled)
